(19) United States
(12) Patent Application Publication
(10) Pub. No.: US 2025/0251331 A1
(43) Pub. Date: Aug. 7, 2025

(54) ORAL NEUTROPHIL ISOLATION AND DETECTION ASSAY FOR PERIODONTAL APPLICATIONS

(71) Applicants: Ebrahim Ghafar-Zadeh, Vaughan (CA); Michael Glogauer, Toronto (CA)

(72) Inventors: Ebrahim Ghafar-Zadeh, Vaughan (CA); Michael Glogauer, Toronto (CA)

(21) Appl. No.: 18/856,844

(22) PCT Filed: Apr. 14, 2023
(86) PCT No.: PCT/CA2023/050512

§ 371 Date: Oct. 14, 2024

Related U.S. Application Data
(60) Provisional application No. 63/330,962, filed on Apr. 14, 2022.

(51) Int. Cl. G01N 15/01; G01N 1/34; G01N 15/1433; G06T 7/00

(52) U.S. Cl. CPC G01N 15/01; G01N 1/34; G01N 15/1433; G06T 7/0012

(57) ABSTRACT

A method of separating neutrophils from a heterogeneous cell suspension comprising the steps of: receiving salivary samples; treating a surface of a receptacle treated with oxygen plasma to increase the hydrophilic properties of the surface that subsequently increase cell-surface adhesion; transferring the salivary samples to the receptacle; adding phosphate-buffered saline (PBS) to levitate epithelial cells and separate them from oral Polymorphonuclear Neutrophils (oPMNs) in saliva; removing the levitated epithelial cells after a delay time (ot), whereby the oPMNs are attached to the surface of the receptacle.

### FIELD

The present disclosure relates to method and systems for measuring oral neutrophils, more particularly it relates to isolating and measuring oral neutrophils in saliva using microscopic images.

### BACKGROUND

Periodontal disease (PD) is a chronic inflammatory condition in which the gingiva, tooth-supporting bone and connective tissue are destroyed. Nowadays, the high prevalence of PDs such as gingivitis and periodontitis in adults has turned it into a public health concern¹; based on the reports of the Centers for Disease Control and Prevention (CDC), at least 47% of the individuals aged 30 years and older are suffering from different forms of periodontal disease, and this percentage experiences a sharp increase to 70.1% in adults equal to and older than 65 years². If not treated or controlled, such periodontal infections result in the destruction of the alveolar bone surrounding the teeth and the periodontal ligament and subsequently tooth loss³. Tooth decay is considered as one of the biggest issues threatening dental health and also one of the important oral health concerns^{2,3}. Periodontal diseases may even affect systemic diseases by both transferring the oral bacteria and toxins in the circulating blood resulting in systemic inflammation and transmitting the orally produced inflammatory factors in the body via the vessels⁴. Periodontal diseases are associated with a large number of other infectious diseases such as diabetes, inflammatory comorbidities, rheumatoid arthritis, and osteoporosis⁵⁻⁸. For instance, PDs increase the risk of cardiovascular diseases, especially in elderly people⁹ when the prevalence of atherosclerotic diseases is higher in individuals with PD⁹. In diabetic cases, the body receives a lower blood amount in the infected tissues compared with non-diabetic individuals, and diabetic have a lower ability to resist infection¹⁰, which increases the risk of tooth loss and PD in diabetic cases. It is known that diabetic people require frequent dental visits and a decrease in the number of visits will result in higher risks of losing any affected teeth. However, a great number of diabetic individuals are not able to visit their dentist in early stages of the oral infections, or they visited too late when the teeth cannot be saved due to the critical damages¹¹. Furthermore, type 2 diabetic individuals with a severe form of PD have higher mortality risk compared with healthy individuals¹⁰. Another consequence of gingivitis is alveolar bone loss that indicates the early stages of osteoporosis. Considering these facts, early detection of the PDs is a crucial step for enabling accurate diagnosis, more definitive treatment and subsequently reducing the risk of other associated diseases.

Researchers have found that neutrophils or so-called oral Polymorphonuclear Neutrophils (oPMNs) in saliva are considered as biomarkers for PDs⁶⁻⁸. Similar efforts have been made to demonstrate the correlation between oPMNs level and severity of gingivitisO. Many other studies have also investigated the association of oPMNs count variations with various diseases or health situations e.g., cardiac bypass¹², bone marrow transplantation¹³⁻¹⁵. Neutropenia¹⁶, neutropenia following chemotherapy¹⁷, oral cancer¹⁸, pregnacy¹⁹, acute prolonged exercise²⁰ and blood engraftment using hematopoietic stem cell transplantation (HSCT)²¹.

Blood PMNs consistently migrate to the oral cavity from blood vessels. Due to the extravasation process, oPMNs are in a higher state of activity compared with blood PMNs which is associated with the presence of some specific cluster of differentiation (CD) marker on their surface. A group of CD markers act as adhesion receptors for neutrophils and control the cell-cell and cell-surface attachments, including CD11a, CD11b, CD18, CD62L, CD66a, and CD66b²². oPMN surface markers contain hydroxyl and methyl groups along with a large number of sialic acids which contribute to several biological features due to their negative charge and hydrophilicity fashion²³⁻²⁵. These proteins have the ability of binding to a wide number of chemical groups which results in high adhesion affinities to the surfaces treated with oxygen plasma or cornstarch⁵⁻²⁷. For this reason, this phenomenon has the potential to be used as an efficient and cost-effective strategy for oPMN isolation by taking advantage of their adhesion properties to different surfaces.

The conventional method for counting oPMNs, as shown in FIG. 1, is to isolate and count oPMN from saliva samples, and includes collecting saliva samples in a test tube (step 1), a filtering process to separate epithelial cells (step 2) and debris (step 3) from the rinsed samples, then measuring the level of oPMNs using automated cell counters²⁸ (step 4). Despite its advantages for periodontal research, this method suffers from some drawbacks. For example, the filtering process used in the conventional oPMN isolation is time-consuming (approximately 3 hours) and substantially inaccurate. In addition, the filtering process requires trained staff, and clinical routine practices involving a large number of human subjects, and therefore it is not suitable for home monitoring²⁹.

### SUMMARY

In one of its aspects, a method of separating neutrophils from a heterogeneous cell suspension comprising the steps of:
receiving salivary samples;
treating a surface of a receptacle treated with oxygen plasma to increase the hydrophilic properties of the surface that subsequently increase cell-surface adhesion;
transferring the salivary samples to the receptacle;
adding phosphate-buffered saline (PBS) to levitate epithelial cells and separate them from oral polymorphonuclear neutrophils (oPMNs) in saliva;
removing the levitated epithelial cells after a delay time (at), whereby the oPMNs are attached to the surface of the receptacle.

In another of its aspects, an automated method of separating neutrophils from a heterogeneous cell suspension comprising the steps of:
receiving a salivary sample in a test tube;
adding phosphate-buffered saline (PBS) to the test tube to create a mixture of the salivary sample and the PBS;
with a programmed microcontroller system, causing a directional pump to turn on and deliver the mixture to a Petri dish, and causing the directional pump to turn off; and after a predefined delay time (σt) causing the directional pump to turn on and remove the solution from the Petri dish and return the solution into the test tube, whereby the neutrophils are attached to the surface of the Petri dish; and wherein the Petri dish is placed on a rotatable platform;
transferring the salivary samples to the test tube;
with the programmed microcontroller system, causing a servomotor to rotate the platform with the Petri dish;
with a microscope and an image capture device, capturing multiple microscopic images as the Petri dish rotates.

In another of its aspects, a system for separating neutrophils from a heterogeneous cell suspension comprising:
a mixture of a salivary sample and phosphate-buffered saline (PBS) in a test tube;
a programmed microcontroller system;
a directional pump controllable by the programmed microcontroller system to turn on and off, and remove the solution from the Petri dish after a predefined delay time (σt), and return the solution into the test tube; and wherein the Petri dish is placed on a rotatable platform;
a servomotor controllable by the programmed microcontroller system to rotate the platform with the Petri dish;
a microscope and an image capture device controllable by the programmed microcontroller system to capturing multiple microscopic images as the Petri dish rotates.

In another of its aspects, a system comprising:
an imaging system for capturing at least one microscopic image of a surface comprising a plurality of isolated neutrophils;
a computer system comprising a hardware processor and a memory device on which instructions are encoded to cause the hardware processor to perform the operations of:
   receiving the at least one microscopic image;
   extracting at least one feature vector set from the image datasets for input into a machine learning architecture;
   generating a machine learning model iteratively trained to detect each of the plurality of isolated neutrophils within the at least one microscopic image;
   annotating the at least one microscopic image by placing a bounding box around and generating image datasets;
   and applying the trained machine learning model to classify each of the plurality of isolated neutrophils appearing within the at least one microscopic image;
   based on the classification, predicting the number of the plurality of isolated neutrophils within the at least one microscopic image.

Advantageously, there is provided a rapid and low-cost oPMN isolation method which is based on a discovery that oPMNs have a strong affinity toward the hydrophilic surfaces in comparison to epithelial cells in the same sample.

Furthermore, there is provided a rapid, accurate, and low-cost cell counting strategy for oPMN detection by adapting state-of-the-art deep learning solutions. The method uses artificial intelligence (AI) techniques for the detection and counting of the cells in saliva from image datasets available related to salivatory cells. The required image datasets are generated for the training of AI-based algorithms for isolating oPMNs from saliva. To date, many studies have reported the detection and counting of biological cells including white blood cells (WBC) and red blood cells (RBC) using various machine learning techniques³⁰⁻³⁴. Unlike other state-of-the-art methods that require several processing steps to detect or classify the objects of interest, the method in this disclosure is an end-to-end intelligent solution based on YOLOv4³⁵, which is capable of providing the results in a single stage and with minimal computational resources and cost compared conventional methods. Accordingly, the method in this disclosure is suitable for real-time point-of-care applications.

The method and system described in this disclosure allows for the isolation and detection of oral neutrophils (oPMNs) in saliva samples for early periodontal disease diagnostic purposes, among others. For example, the functionality and applicability of this rapid (less than 15 minutes) and low-cost isolation method is confirmed by studying various factors including the hydrophilicity of a surface created by oxygen plasma and corn-starch. In addition, a deep learning-based framework is employed to detect oPMNs in saliva samples, in which YOLOv4 is adapted to achieve high accuracy and precision (90% and 95% respectively), in a fraction of second runtime. The framework can be deployed on a cloud server that can also be employed for archiving the microscopic images and further analysis and future model parameter updates.

Furthermore, the method and system described in this disclosure is used to extract other saliva cells such as epithelial cells for understanding the correlation between various diseases and saliva cells.

In another aspect, the method and system described in this disclosure may be employed to develop a low-cost handheld oPMN isolation and detection using smartphones for the early detection of periodontal diseases at home; which has the promise of opening a new avenue in the future to widely employ saliva for disease diagnostics.

### BRIEF DESCRIPTION OF THE DRAWINGS

FIG. 1 shows a prior art method for isolating oPMNs from saliva samples;

FIG. 2 shows a method for counting of oPMN from saliva samples;

FIG. 3a shows forces acting on neutrophil and epithelial cells in a first phase of an isolation protocol, in which the cells are floating and are subjected to gravity and the fluid drag force added to buoyancy force that act in the opposite direction of cells sedimentation;

FIG. 3b shows forces acting on neutrophil and epithelial cells in a second phase of the isolation protocol, in which the cells undergo a suction force from a pipette, and as a result, the neutrophil and epithelial cells experience lift, drag from fluid and adhesion forces from surface of a Petri dish;

FIG. 4a shows an automated system for isolating neutrophils;

FIG. 4b shows YOLOv4 overall structure comprising: (portion (a)) Backbone: CSPDarkNet-53; (portion (b)) Neck: SPP; (portion (c)) Neck: PANet; and (portion (d)) Head: Dense prediction from three different scales;

FIG. 5a shows a COMSOL simulation result: (a) falling velocity of neutrophil and epithelial cells based on the modeling;

FIG. 5b shows a COMSOL simulation result: (b) suction of a cylinder cut of the Petri dish containing the saliva and PBS;

FIGS. 6a-c show COMSOL simulation results of shear rate of saliva on the surface of Petri-dish; (a) shear rate for the condition of mass flow rate 200 μL/min, (b) shear stress contours generated due to pipetting saliva into the Petri dish; the locations A, B, C, and D show the lowest shear stress on the bottom of Petri dish; (c) profile of shear stress (Pa) along the red dotted lines in (b), showing the decay of shear stress from the point of injection to the Petri dish wall;

FIG. 7 shows fluorescence microscopic images of saliva sample (a-b) single epithelial cells, (c) both epithelial cells and oPMNs and (d-e) single oPMN with a three-segment nucleoli (X40);

FIG. 8a shows light microscope images of the oPMNs before applying the isolation method (X40);

FIG. 8b shows light microscope images of the oPMNs after applying the isolation method (X40);

FIG. 9a shows an oPMN isolation method (i) before and (ii) after applying the isolation method in order to study the role of oxygen plasma;

FIG. 9b shows an oPMN isolation method (i) before and (ii) after applying the isolation method in order to study the role of delay o/swashing time;

FIG. 9c shows an oPMN isolation method (i) before and (ii) after applying the isolation method in order to study the role of CaCl₂;

FIG. 9d shows an oPMN isolation method (i) before and (ii) after applying the isolation method in order to study the role of PBS;

FIG. 9e shows an oPMN isolation method (i) before and (ii) after applying the isolation method in order to study the role of con starch;

FIG. 9f shows an oPMN isolation method (i) before and (ii) after applying the isolation method in order to study the role of the type of surface materials;

FIG. 10 shows linearity of the oPMN isolation method, in which the number of the cells in each spot after isolation versus number of the cells before isolation, in which the initial number of cells (˜18 cells/spot) per spot was multiplied by a factor between 0.25 to 2;

FIGS. 11a-c show three examples of the algorithm performance, in which FIG. 11a shows original images, FIG. 11b is the annotated data, and FIG. 11c depicts the outcome of the implemented CNN method;

FIG. 12 shows an algorithm performance on the 40 never-seen test dataset; in which true positives (TP) are shown in blue, false positives (FP) are shown in orange, and false negatives (FN) are demonstrated in gray color;

FIGS. 13a-c show neutrophils attached together; and FIGS. 13d-e show superimposition of different biological cells (e.g. epithelial cells and neutrophils);

FIG. 14 shows oPMNs detected with a 10X objective using fluorescent and light microscopy in two different predefined time periods;

FIG. 15 shows oPMNs detected with a 20X objective using fluorescent and light microscopy in two different predefined time periods; and

FIG. 16 shows oPMNs detecting with a 40X objective using fluorescent and light microscopy in two different predefined time periods.

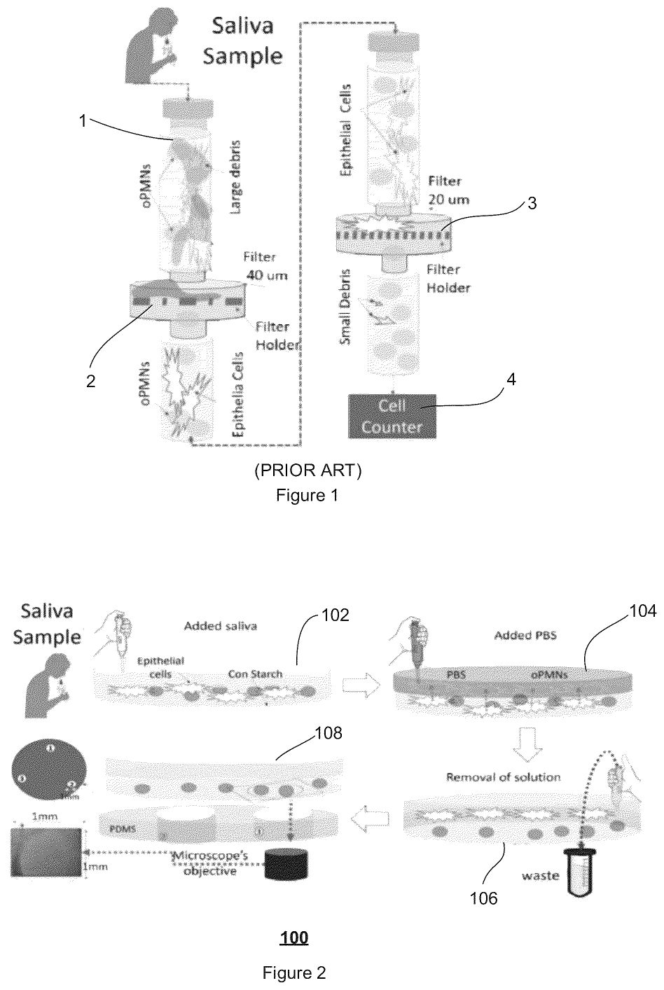

### DETAILED DESCRIPTION OF EXEMPLARY EMBODIMENTS

The following detailed description refers to the accompanying drawings. Wherever possible, the same reference numbers are used in the drawings and the following description to refer to the same or similar elements. While embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims.

Moreover, it should be appreciated that the particular implementations shown and described herein are illustrative of the invention and are not intended to otherwise limit the scope of the invention in any way. Indeed, for the sake of brevity, certain sub-components of the individual operating components, and other functional aspects of the systems may not be described in detail herein. Furthermore, the connecting lines shown in the various figures contained herein are intended to represent exemplary functional relationships and/or physical couplings between the various elements. It should be noted that many alternative or additional functional relationships or physical connections may be present in a practical system.

Referring to FIG. 2, there is shown a flow diagram 100 outlining exemplary steps for isolating oPMNs details of which are discussed in detail below.

Sampling Procedure and Study Participants In one study, participants were informed not to drink (except water) and eat two hours before the experiments. Oral rinse samples were taken from all subjects and the oPMNs were isolated on the same day right after the sampling procedure. A 3 mL sterile sodium chloride solution (0.9% NaCl) was used by each participant to serially rinse their mouth five times. After oral rinsing, 15 mL salivary samples were obtained which included both epithelial cells and oPMNs. In the study, the participants were volunteers aged between 18-60 years old, both male and female. The purpose of the study and the protocols were explained to each subject, and they received written information and written informed consent was obtained from all participants in advance.

oPMNs Isolation Biological Protocols

The protocols included oPMNs sampling, isolation and microscopy, and surface treatment using the corn-starch and oxygen plasma, and the conventional methods for isolation and counting of cells using filtering and hemocytometry-based techniques, respectively.

oPMNs Isolation

oPMNs were separated from the saliva samples in 4 steps, as seen in FIG. 2. In step 102, each oral rinse sample taken from the subjects was transferred to two different Petri-dishes treated with oxygen plasma and corn-starch. In the first attempt, the salivary samples were introduced to the oxygen plasma-treated glass Petri dish which was followed by adding phosphate-buffered saline (PBS) to levitate the epithelial cells and separate them from oPMNs (step 104). The oxygen plasma (70% power intensity) significantly increases the adhesion of oPMNs surrounded by hydrophilic CD markers²². PBS was used to wash and weaken the bonding between epithelial cells or debris and the surface of the Petri-dishes. PBS was added with the aim of removing the dead, non-adhesive, and lose cells as well as the cellular particles^{36,37}. In step 106, after a delay time (σt) allowing the settling of oPMNs, the levitated epithelial cells are removed in the third step using a micropipette while the oPMNs are attached to the hydrophilic surface. Alternatively, another technique to increase the hydrophilic properties of the surface that subsequently increase the cell-surface adhesion. In this technique, the surface of the glass was treated with corn-starch³⁸⁻⁴⁰ as described in the related protocol. The oPMNs are expected to remain attached on the surface of glass Petri-dish.

To characterize and set up the proposed isolation method, the effect of different effective parameters including the delay time, oxygen plasma, corn-starch, CaCl2, PBS, and surface materials were also investigated on the adhesion properties of the neutrophils and the count of the isolated oPMNs.

### Microscopic Imaging

In this protocol, in order to reduce the error of the non-uniform distribution of oPMNs on the Petri-dish, three microscopic images are captured (step 108). The mean values of numbers of oPMN detected in each spot will be used in equation (1) to calculate the oPMNs level in each sample. For this purpose, a Polydimethylsiloxane (PDMS, Dow curing Sylgrad 184 silicone, Fisher Scientific Canada) membrane (10 cm diameter, 1 mm thickness) with three holes (1 mm diameter) was prepared to ensure the consistency of measurements. In one example, isolated oPMNs are detected under a 40× magnification on a light microscope (e.g. Olympus IMS). The microscope is focused on each one of the circular areas (1-3) with ¬1 mm diameter to capture three images.

### Oxygen Plasma Treatment

In more detail, the oxygen plasma significantly increases the adhesion of oPMNs surrounded by hydrophilic CD markers. For this reason, the glass Petri dishes were first cleaned with 70% ethanol and then air-dried. This step was carried out in a clean room with no dust particles to make sure the surfaces of the Petri dishes were clean for activation. A plasma surface treatment machine (e.g. Zepto LF PC—Diener electronic) was used and set up for 70% power. The Petri-dishes were put inside the machine and when the pressure reached 0.2 mbar, the plasma activation was turned on. The duration of the exposure was greater than 5 minutes. After the treatment, the Petri-dishes were sealed and kept in a sterile environment prior to the experiments.

### Corn-starch Preparation and Surface Treatment

The corn-starch (e.g. from Sigma-Aldrich Canada) was prepared and coated on the sterile glass Petri-dishes in six steps as follows. Dry and pure starch (10 mg, dry solids) was added to a glass vial and 20 mL ethanol was added to wet the starch. Then, 5 mL of 90% DMSO (Sigma-Aldrich Canada) was added to cover the glass vial containing the sample. Subsequently, the tube containing the mixture was boiled for 1 hour in a boiling water bath while magnetic stirring. Thereafter, a magnetic stirrer was used to mildly stir (at 155 RPM) the starch solution up to 24 hours at room temperature. Next, 600 μL of the solution was added to the Petri-dishes and it was assured that the corn-starch had covered the surface of the Petri dishes completely. Eventually, the modified Petri-dishes were allowed to dry at room temperature for one hour prior to the experiments.

### Fluorescence Staining

The oPMNs recovered from both the novel oPMN isolation described herein and the conventional isolation were quantified using fluorescence staining to evaluate the accuracy of the oPMN counting using light microscopy. For fluorescent quantification, firstly 100 μL of 10% formaldehyde (Sigma-Aldrich Canada) was added for fixation of the cells on the slides. After a 15-minute wait, 1 μL acridine orange was diluted with 99 μL PBS and the obtained 100 μL solution was added to the slides containing the fixed cells and waited for another 15 minutes. The imaging and further quantification were performed using a fluorescent microscope (Olympus BX50) using a 20× long focal length objective lens and 10× ocular lenses. The orange granular fluorescence was used to detect the oPMN cells. The oPMNs were counted in 10 different microscopic areas and their concentration in the oral rinse sample was obtained for both novel and conventional isolation methods.

Conventlesal oPMNs Isolation

A conventional filter-based oPMN isolation process was also used as a control for the proposed oPMN strategy. For this purpose, a 5 mL sterile sodium chloride solution (0.9% NaCl, Sigma-Aldrich Canada) was used by each participant to serially rinse their mouth 10 times. After oral rinsing, 50 mL salivary samples were obtained from the participants which included both epithelial cells and oPMNs. The obtained samples were distributed into two new 50 mL falcon tubes (25 mL in each) via 40 μm filters. Afterward, the samples were transferred into two 25 mL syringes and the whole filtered 40 mL samples were gently inserted in four new tubes using 11 μm filters. Then, the tubes containing 10 mL samples were centrifuged at 2600 RPM for 5 minutes at 4° C., the supernatants were discarded and 1 mL sample was left in each tube. The remaining 1 mL samples were transferred into new tubes. The solutions in these new tubes were expected to include pure oPMNs. 10 μl of the oPMN samples were used to evaluate the concentration of the oPMNs in each sample via a Hemocytometer under 40× magnification on a light microscope.

### Linearity Characterization

The linearity of the proposed isolation protocol was characterized using saliva samples with different known input concentrations (CI) and concentrations of output isolated oPMNs (CO). it was assumed that the isolation efficiency (CO/CI) is equal to λ. Then, different values of COs were prepared by using centrifuge and dilution processes. For instance, 0.5×CI was achieved by adding normal saline with the same volume of a saliva sample, or 2×CI was achieved by centrifuging the sample and discarding half of the normal saline from the sample. With these sample protocols, test samples were prepared with different concentrations including 0.25×CI, 0.5×CI, CI, 2×CI and 4×CI; the isolation procedure was repeated at least three times for each dilution and the obtained oPMN concentrations were analyzed for the linearity of the relationship between the salivary oPMN dilution and oPMN isolation count.

oPMNs Isolation Mechanism

A critical observation in this study was the rapid settlement and adhesion of the oPMNs on the surface. Herein, the probable causes for this interesting phenomenon are put forward.

### Role of CD Markers

Neutrophils have a remarkable tendency to interact with non-physiologic surfaces, e.g., plastic, nylon, and glass. Different CD markers on the neutrophil surface ae responsible for cell attachment, especially CD11b and CD18⁴¹⁻⁴⁵. The hydrogen bonding between the hydroxyl groups of these highly glycosylated CD markers and the hydroxyl groups on the oxygen plasma- or com-starch-treated glass surfaces might be the reason for this observation. Facing these surfaces activates PMNs and upregulates CD11b and CD18 expression which boosts cell-surface adhesion⁴⁶. On the other hand, the activated oPMNs have frequent negatively charged sialic acids on their surfaces resulting in an intense affinity to the hydrophilic surface with numerous hydroxyl groups⁴⁷. All of these features along with the delicate molecular structures make oPMNs capable of binding to a hydrophilic surface strongly.

### Hydrodynamic Forces

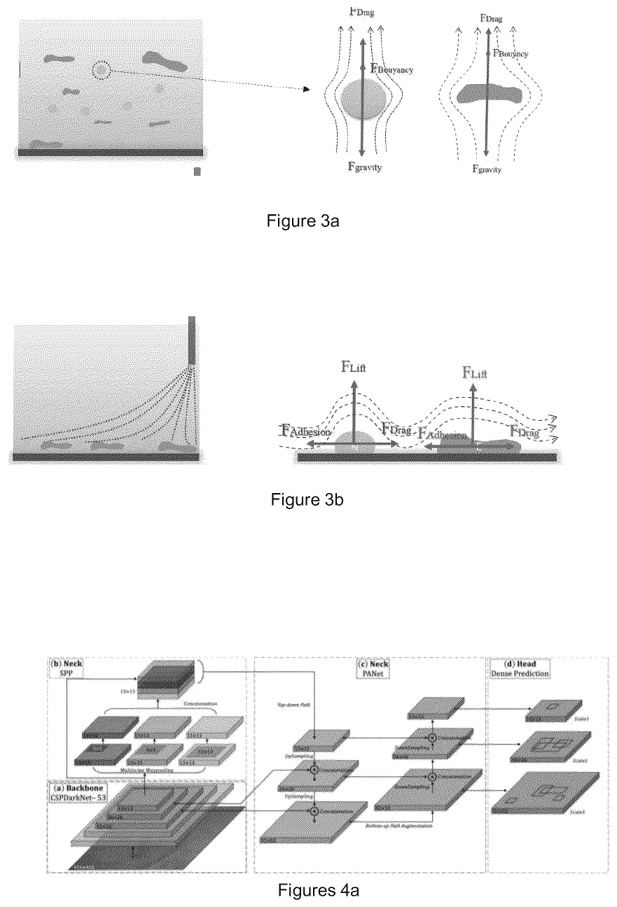

The physical forces applied on the saliva cells are shown in FIGS. 3a and 3b for two phases of the presented isolation protocol. As shown in FIG. 3a, the saliva sample is injected into the Petri-dish, (phase 1), and then the waste sample is sucked out (phase 2) as shown in FIG. 3b. When cells are left free to be float in the medium before reaching the surface, the major forces acting on them could be defined as gravity (FG), buoyancy (FB), and drag (FD) forces. The FG roots from the mass of the cell which pulls it towards the surface of Petri-dish. The FB (FBouyancy) is inherently proportional to the volume of cells multiplied by the density of saliva (buoyancy force). According to FIG. 3, FD (FDrag) is proportional to the resistance of the fluid to the motion of cells that play an important role in isolation protocol. During the free-fall phase, the relation FD+FB−FG=ma describes the motion of cells, where m and a represent the mass and acceleration of the cells, respectively. Due to the significant difference in the shape and density of oPMNs and epithelial cells during the second phase, epithelial cells experience higher FD (drag force) and consequently obtain lower velocity in the first phase. Furthermore, the sudden change in density of fluid due to the increase of salt concentration, [48], or pH would push the cells to detach from the surface and float due to the superior proportion of FB to gravity and drag. This was put under further investigation by performing the COMSOL fluidics simulation to better demonstrate and discuss the underlying mechanisms of the proposed isolation process.

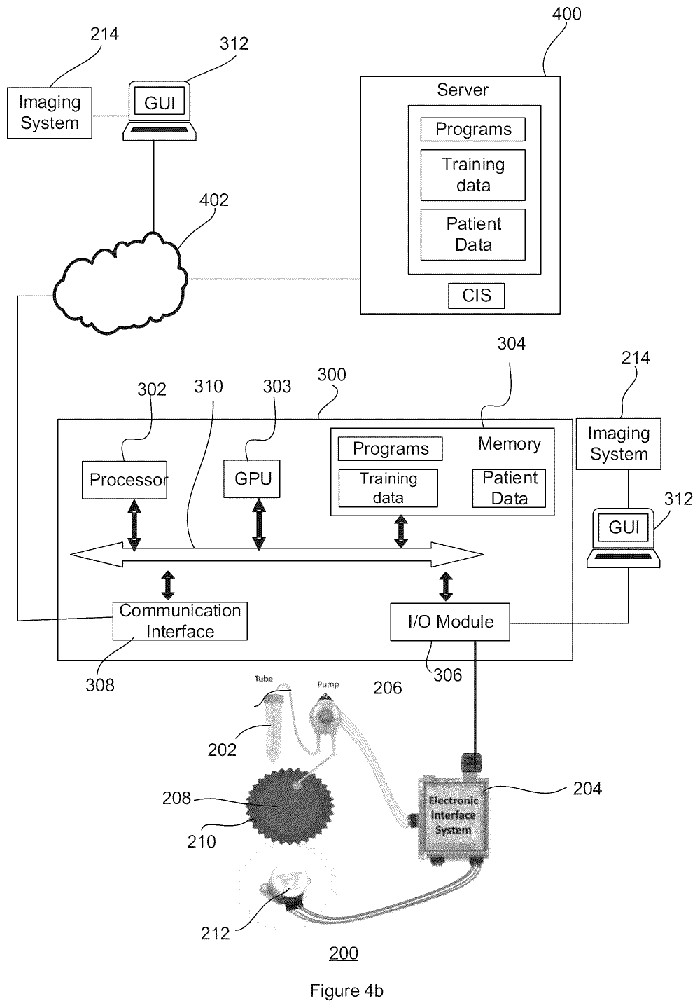

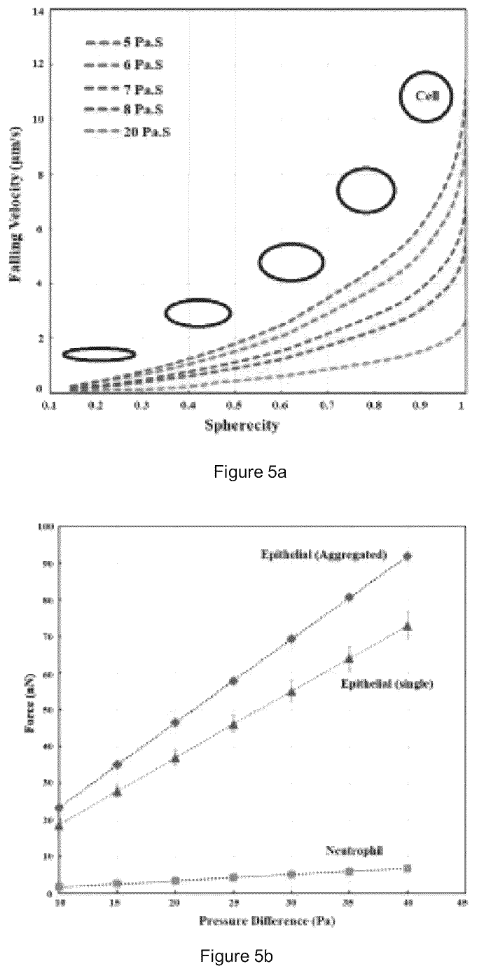

FIG. 4a shows an automated system 200 for isolating neutrophils. The system 200 comprises a test tube 202 with a salivary sample and phosphate-buffered saline (PBS), a programmed microcontroller system 204, causing a directional pump directional pump 206 to turn on and deliver the mixture to a Petri dish 208, and causing the directional pump 206 to turn off: and after a predefined delay time (ot) causing the directional pump 206 to turn on and remove the solution from the Petri dish 208 and return the solution into the test tube 202, whereby the neutrophils are attached to the surface of the Petri dish 208; and wherein the Petri dish 208 is placed on a rotatable platform 210; transferring the salivary samples to the Petri dish 208; with the programmed microcontroller system 204, causing a servomotor 212 to rotate the platform 210 with the Petri dish 208; with an imaging system 214 for capturing multiple microscopic images comprising a plurality of isolated neutrophils as the Petri dish 218 rotates.

The imaging system 214 is communicatively coupled a computer system 300 comprising a processor 302, a general processing unit 303, a memory device, such as memory 304, an input/output (I/O) module 306 and a communication interface 308, which are in communication with each other via centralized circuit system 310. A graphical user interface (GUI) 312 may be coupled to the computing device 300 via the input/output (I/O) module 306. Stored on the memory device 304 are instructions encoded to cause the hardware processor 302 to perform the operations of receiving the at least one microscopic image; extracting at least one feature vector set from the image datasets for input into a machine learning architecture; generating a machine learning model iteratively trained to detect each of the plurality of isolated neutrophils within the at least one microscopic image; annotating the at least one microscopic image by placing a bounding box around and generating image datasets; and applying the trained machine learning model to classify each of the plurality of isolated neutrophils appearing within the at least one microscopic image; based on the classification, predicting the number of the plurality of isolated neutrophils within the at least one microscopic image.

oPMNs Detection

A deep learning technique along with the developed image dataset is used to count the number of oPMNs in each spot.

### Image Dataset

In more detail, after sample preparation, as described above, 226 saliva microscopic images were captured using microscopic imaging techniques described in the above subsections. These images were annotated using the graphical image annotation tool in 48. The cohort of microscopic images comprises 4,617 oPMNs, in which train, validation, and test datasets were selected randomly. In each raw microscopic image within the dataset, oPMNs were confined with an appropriate bounding box and the results were saved in YOLO format. In each microscopic image, neutrophils are the objects, while the remaining structures including epithelial cells are considered as the background. Alternatively, fluorescence microscopy was also performed, as seen in the results, to prove that the saliva sample only includes oPMNs, epithelial cells, and debris that can be observed in the microscopic images.

### Deep Learning Method

YOLO family algorithms⁴⁹⁻⁵², in general, unify the regression and classification tasks into a single convolutional neural network (CNN) architecture. Each given bounding box includes the class probability and comprises the regression problem information such as the location and size of the object of interest (x, y, w, h). This single-shot execution strategy enables an optimized end-to-end model training and allows the network to process images at a very high frame-per-second (FPS). In conventional YOLO algorithms⁴⁹⁻⁵⁰, predictions are made at single granularity level, which restricts the network from finding smaller objects. Recent YOLO versions including YOLOv3⁵¹, and the selected YOLOv4⁵² have adapted multi-scale target detection concepts from other AI frameworks to facilitate information flow between different network layers. These algorithms use strategies such as feature pyramid network (FPN)⁵³ and PANet⁵⁴, which result in a better performance in detecting small and overlapping objects. YOLOv4 architecture is built by three main blocks namely, Backbone, Neck, and Head, as shown in FIG. 4b. Backbone in the network is responsible for feature extraction (a) and is typically based on CSPDarknet53 or CSPResNeXt50⁵⁵. Due to the better performance of CSPDarknet53 as presented in the original YOLOv4 paper, it is appointed as the backbone network in this study. The neck in the network combines features from the backbone and makes them prepared for the detection step (b-c). It is designed to mix information from the contracting (spatial rich) and expanding (context-rich) blocks of the network, and then propagate the resulted features to the detection part of the model. The head, on the other hand, enables a one-stage object detection for dense prediction (d). This part provides a vector that comprises information regarding the class label, prediction confidence score, and the bounding box information (x, y, w, h).

Compared to precedent YOLO architectures, YOLOv4 also applies two model training modification strategies namely bag of freebies (BoF) and a bag of specials (BoS), that can improve the overall performance even having a modest amount of training dataset. BoF is composed of the strategies such as data augmentation, loss function modification, and hyperparameter tuning that in general do not influence the algorithm testing FPS and the only cost is during the training phase; thus, it is an off-line procedure. In contrast, BoS strategies improve the performance by model architecture redesign, though this has a minor impact on the runtime of the algorithm.

### Optimization and Quantification Methods

In order to optimize the performance of the algorithm, transfer-learning was used and the weights of a pre-trained model on MS COCO dataset³⁵ were used for initializing the model's backbone weights. Compared to the randomly initialized network, it is hypothesized to achieve higher precision. The precision, sensitivity and F₁ score are the used metrics to evaluate the AI solution, which are defined in the equations (1)-(3).

$$Sensitivity = \frac{TP}{T P + F N} \qquad ( 1 )$$

$$Presicion = \frac{T P}{TP + FP} \qquad ( 2 )$$

$${F}_{1} score = 2 \times \frac{Sensitivity \times Presicion}{Sensitivity + Presicion} \qquad ( 3 )$$

Where TP=true positive, FP=false positive, and FN=false negative. The sensitivity metric in equation (1) provides a quantitative measurement of detected neutrophils, while precision gives a measure of the detection quality. A higher level of TPs is associated with higher sensitivity, whereas a smaller number of FPs determines the precision of the algorithm. F₁ score is the harmonic mean of sensitivity and precision and gives a measure of accuracy and the balance between two metrics. Also, the class label probabilities above 50% are preserved to limit the number of false positives. Overall, there is a trade-off between increasing the TP rate and decreasing the number of FP s. Based on experience, 50% confidence holds the balance between these two parameters.

### Results and Discussions

The results related to the isolation of neutrophils from saliva samples and the deep learning detection will now be described.

### COMSOL Simulations

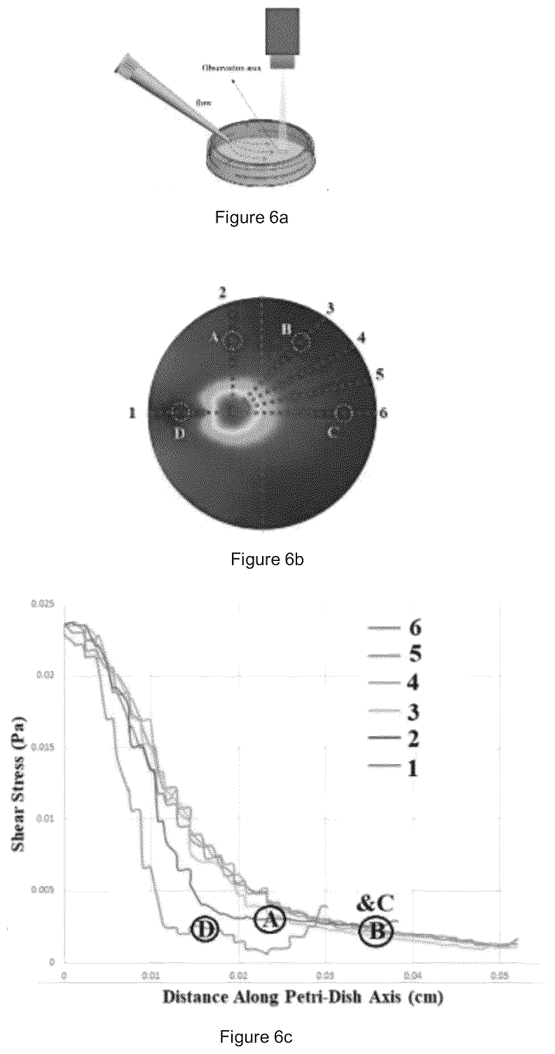

To understand the physics behind the sedimentation of epithelial and neutrophil cells, a COMSOL Multiphysics simulation is performed to model the process. As shown in Figure Sa, the COMSOL simulations results show the falling velocity of epithelial cells in the first phase of the proposed isolation method. Knowing that the epithelial cells and oPMNs have non-spherical and almost spherical shapes respectively, simulation results demonstrate that the falling velocity of oPMNs is greater than epithelial cells ending up faster sedimentation of oPMNs. Additionally, in the second phase, simulations clarified that pressure differences caused by the suction create lower forces on the oPMNs than epithelial cells during the suction, as seen in FIG. 3b. Herein, another simulation was performed to find the best locations for the observation of cells. As shown in FIG. 6, the shear stress created during the injection of the saliva sample or PBS decay to zero in the locations of A-D near to the wall of Petri-dish; therefore, these or other locations with higher distances from the center of injection of the sample can be considered the optimal location of taking the microscopic images in the second phase with less movement of oPMNs.

### Experimental Results

oPMN Isolation

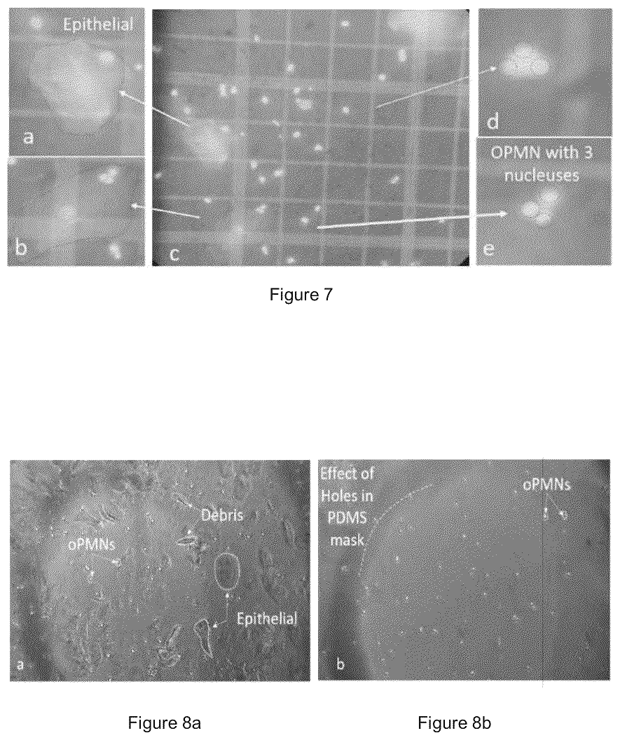

The isolation method described in this disclosure was successfully applied on more than 37 saliva samples to isolate oPMNs from the saliva. The saliva sample includes epithelial cells, oPMNs, and debris, as shown in FIG. 7 and FIGS. 8a-8b. This method was used to remove epithelial cells and debris as seen in FIG. 8a-8b. FIG. 7 show fluorescence images as well as the light microscopic images of the sample before and after isolation, as shown in FIGS. 8a-8b. In light microscopy images, the circular shape of holes in the PDMS can also be seen in the images. As seen in these figures, the proposed method could effectively remove the debris as well as epithelial cells from the Petri-dish.

### Characterization and Optimization

Generally, the performance of the isolation method is a function of several parameters including delay time (at), the exposure time in the oxygen plasma (tO.P) or the concentration of corn-starch (CC.S), the percentage of PBS (PPBS) in the mixture of water and PBS, the concentration of CaCl₂ that is routinely used to activate the cells (CCaCl₂). Additionally, knowing a number of oPMNs with weaker adhesion property might be removed from the sample in step 4 (see FIG. 2c) of the process, the number of oPMNs before and after isolation is not expected to be the same; however, a linear relationship between the number of oPMNs before and after isolation allows us to multiply the number of isolated oPMNs with a constant coefficient; and obtain the actual number of oPMNs in the sample.

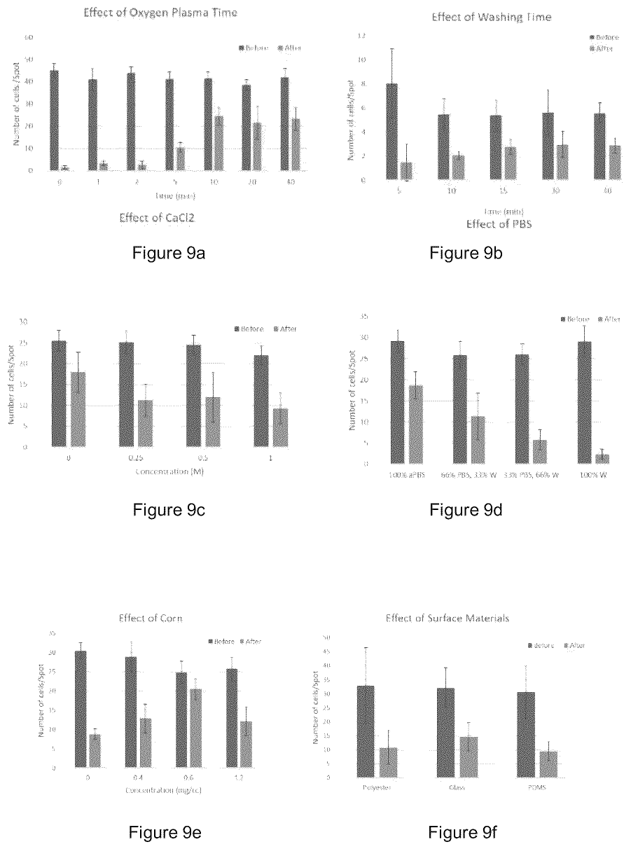

Various protocols were performed to characterize the proposed isolation method by studying the effect of various parameters including the delay time, oxygen plasma, corn-starch, CaCl2, PBS, and surface materials as shown in FIGS. 9a-f. In each diagram, 3 to 7 different samples were used that are equal to the number of blue (or red) columns shown in FIGS. 9a-f. Each saliva sample taken from a human subject might have a different oPMN. Therefore, the effect of the aforementioned parameters cannot be verified by comparing the number of oPMNs. For this reason, the conventional filtering and hemocytometry methods were used to count the equivalent number of oPMNs in each sample. Therefore, in the proposed protocol, the number of oPMNs detected in the ith sample (Ni, 1<i<4) was multiplied by the N1/Ni. For each column in FIGS. 9a-f, three experiments were repeated using the same saliva sample. In each experiment, the different numbers of oPMNs were read out using the microscopic images taken from three different spots in the Petri-dish. Therefore, each blue or red column in each diagram was created using nine measurements that are the numbers of oPMNs before and after the isolation. The error bar in each column is equal to the standard deviation of the nine numbers. FIG. 9a reveals the effect of exposure time in increasing the hydrophilicity and correspondingly the adhesion and increase of the output. Based on this result, exposure times greater than 5 minutes can effectively increase the number of attached oPMNs. FIG. 9b shows that by increasing the delay time, higher than 10 minutes, the percentage of output is increased. Based on these results, more than 15 minutes delay will not increase the output and the curve reaches a saturation state. Indeed, this delay time is required to allow that the epithelial cells are levitated and the oPMNs have sufficient time to be attached on the surface.

As seen in FIG. 9c, CaCl₂) did not show any increase in the adhesion of oPMNs. Indeed, the adhesion of oPMNs on the surface is because of a physical hydrophilic property of cells and likely CaCl₂ cannot increase the hydrophilic property. Another reason may be that the oPMNs are already activated during their migration toward the oral cavity; hence, adding CaCl₂ would not increase the state of their activation anymore. Also, unlike other cell-surface interactions, the attachment of cells will not create any focal adhesion because in this isolation protocol, instead of culture medium and other growth factors, normal saline or PBS are used that cannot result in the growth or proliferation of cells. FIG. 9d shows the effect of the percentage of PBS in a mixture with deionized (DI) water. Adhesion properties of the cells highly depend on the pH of the solution. Attachment of the cells to a surface requires specific pH and concentrations of Mg²⁺ or Ca²⁺. Therefore, when PBS lacks magnesium or calcium, the adhesiveness of the cells decreases⁵⁶. As seen in FIG. 9d, the higher percentage of PBS can effectively separate the non-bonded cells and debris and accordingly increase the percentage of oPMNs on the surface. In this characterization process, the effect of CaCl₂ was also studied, CaCl₂ is known as an agent for activating cells and increasing cellular adhesion by activating specific internal signaling pathways⁵⁷⁻⁵⁹

In FIG. 9e, the effect of corn-starch with different concentrations was studied. Starch-based materials have been used in biomedical investigations for different purposes due to many advantages especially their biocompatibility and no immunogenic effect on the leukocyte⁶⁰⁻⁶². As demonstrated in FIG. 9e, by increasing the concentration of corn-starch, the adhesion properties are enhanced, and consequently, the output is increased. Finally, as seen in FIG. 9f, the effect of three different surface materials including PDMS, glass, and polystyrene was investigated. As this figure depicts, the glass surface with higher hydrophilic properties demonstrates higher adhesion properties.

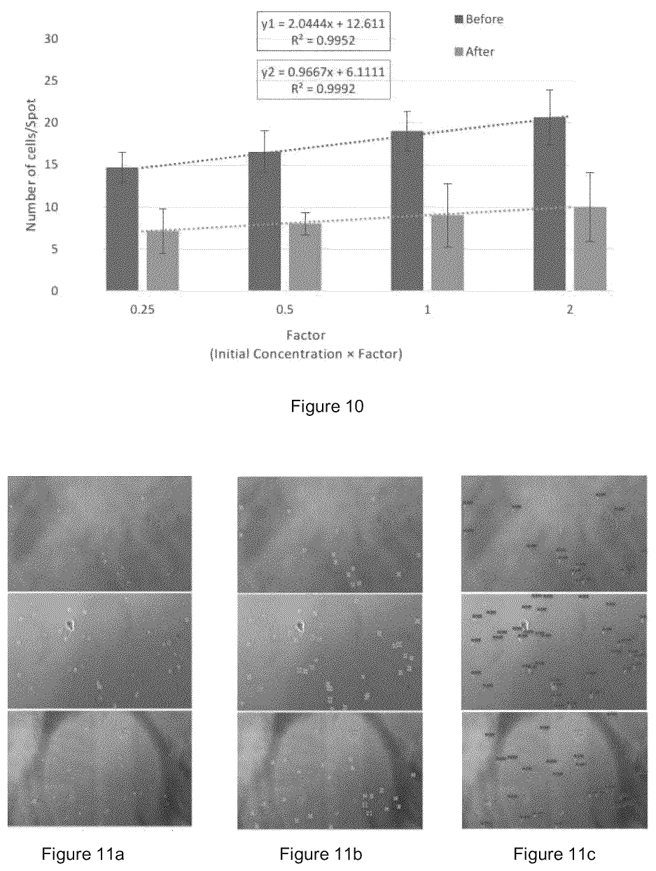

In another effort, the relationship between the number of oPMNs before and after isolation was studied. In this study, four different concentrations from the initial concentration were prepared as aforementioned in the related protocol. As seen in FIG. 10, the number of isolated oPMNs in each spot is linearly increased by increasing the concentration of oPMNs in the saliva sample. The relationship between the number of oPMNs before (y1) and after (y2) isolation can be shown in equation (4) using the calculated relationships in FIG. 10.

$${y}_{2} = 0.472 {y}_{1} - 0.0574 \qquad ( 4 )$$

In this equation, the slope of this linear relationship is κ=0.472. It is noteworthy u can be varied by changing the isolation conditions such as the delay time, oxygen plasma exposure time, etc. Therefore, assuming the volume of the saliva sample is 1 mL, the concentration of oPMNs (C) in the saliva sample can be obtained using the following equation.

$$C = \frac{{D}^{2}}{κ} {y}_{2} \qquad ( 5 )$$

where D is the diameter of Petri-dish. Indeed, the total number of oPMNs is equal to the number of oPMNs observed in each spot with 1 mm² surface area multiplied by the total surface area. For example, in FIG. 9a, the concentration of the sample with 10 minutes delay will be equal to 0.472×(100²)×25=118 Kcells/mL.

### Deep Learning Detection and Counting

The algorithm, described above, was trained over 9,000 iterations and the best performing model on the validation dataset was used to compute the test accuracy. The used dataset details for training and inference of the deep learning solution is brought in Table 1. The results of the algorithm on the test dataset including sensitivity, precision and F₁ score are reported in Table 2.

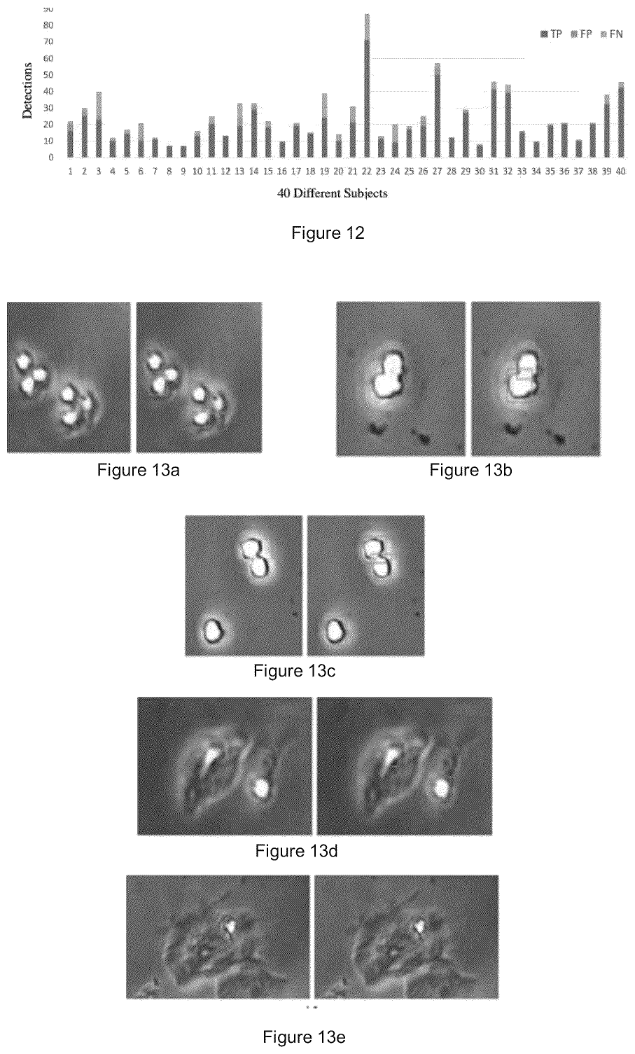

To reveal the functionality of the implemented deep learning framework, as examples, three different images were chosen from the test set, as shown in FIG. 11a. Then, the algorithm's results together with the ground truth were shown in FIG. 11c and FIG. 11b, respectively. FIG. 11c depicts the detected oPMNs after applying the deep learning algorithm. Further to the reported average values in Table 2, FIG. 12 shows the model's performance on individual test images.

TABLE 1
Training, Validation, and Testing Datasets Characteristics

| Database | Images | Neutrophils |
|---|---|---|
| Train | 146 | 2,867 |
| Validation | 40 | 810 |
| Test | 40 | 940 |

TABLE 2
Results of the Algorithm on 40 Different Test Images

| Sensitivity | Precision | F₁ score |
|---|---|---|
| 85% | 95% | 90% |

As seen in FIG. 12, there is only a limited number of outliers in the results that show the model's robustness confronting the challenging task of neutrophil detection from raw saliva microscopic images.

As illustrated in portion (d) of FIG. 4a, with an input image of size 416×416, the model's three detection layer sizes are 52×52, 26×26, and 13×13, which means feature vectors are down-sampled 8×, 16×, 32×, respectively. In other words, using multiscale Head strategy (portion (d) of FIG. 4a) objects above 8>×8 pixels are detected. Also, overlapping structures whose centers are more than 8 pixels apart should theoretically be detected. As it can be inferred, multiscale detection strategy can help handling the challenge of detecting varying size and shape objects, and the ones which are in close proximity. The results show that not only YOLOv4 can be used to detect small objects, but it can also tackle the detection problem of overlapping structures, as can be seen in FIGS. 13a-c.

Another challenge is the superimposition of different cells within the images. The most frequent state is the overlap between epithelial cells and neutrophils. FIGS. 13d-e demonstrate the robustness of the proposed algorithm in separating neutrophils from other biological cells in the microscopic images. Unlike many previous state-of-the-art works that for the sake of simplification, use preprocessed and cropped microscopic images containing a single or limited number of cells in the field of view (FOV) 30-34, the method and system in this disclosure used raw images without any pre- or post-processing. As such, advantageously, the platform described in this disclosure allows real-time image processing with low computational costs at point-of-care setups. For example, using an NVIDIA Tesla T4 GPU 303 with 16 GB of memory and an Intel® Xeon® 2.20 GHz CPU, the time to run the model on a test dataset is approximately 33 milliseconds. On the other hand, deep intuition added through the choice of AI solution helps us to surmount the shortages in database quantity and quality. Unlike conventional methods and systems, the platform described in this disclosure does not require highly magnified, very good quality, or even stained images. As a result, the platform described in this disclosure is suitable for handheld device applications that in comparison with clinical setups provide inferior image qualities.

Annotation Rules using Fluorescent microscopy

Training AI with microscopic images presents a challenge in terms of annotation, as the presence of each oral neutrophil cell must be detected despite their varying morphology over time. To address this challenge, there is provided an annotation method that utilizes fluorescence images to identify cells and then matches them to their corresponding cells in optical microscopic images, thereby allowing for a set of rules for substantially accurate annotation to be generated.

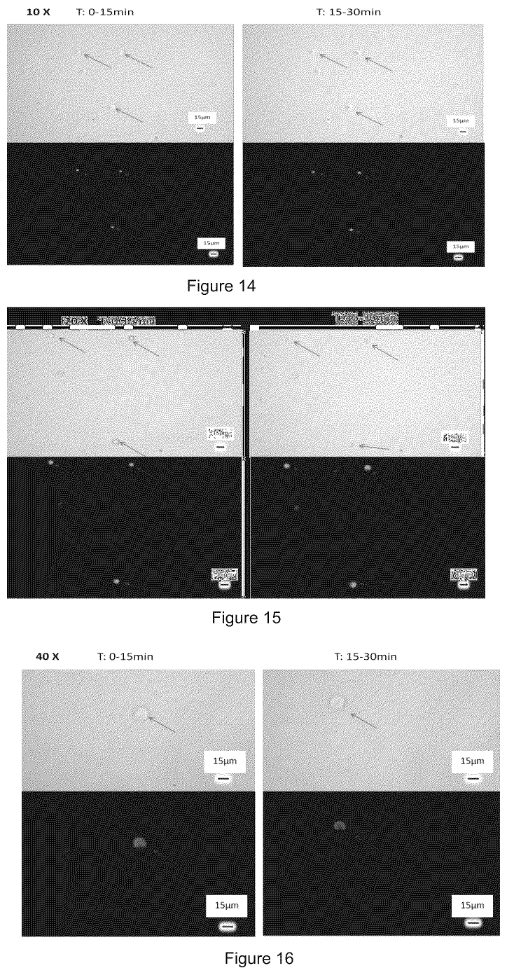

In one example, fluorescent identification is used to demonstrate the morphology and structure of neutrophil cells in saliva. By annotating these cells using 10×, 20×, and 40× fluorescent and light microscopy, rules for training the AI-based algorithms are developed. Through examination of the shapes and morphological structures of the stained nucleus in fluorescent microscopy, the oral neutrophil cells were identified. For example, the images were taken during two different time durations: 0-15 and 15-30. FIG. 14 shows oPMNs detected with a 10X objective using fluorescent and light microscopy in two different predefined time periods; FIG. 15 shows oPMNs detected with a 20X objective using fluorescent and light microscopy in two different predefined time periods; and FIG. 16 shows oPMNs detecting with a 40X objective using fluorescent and light microscopy in two different predefined time periods.

Table 3 shows rules for training the deep-learning framework and annotating the automated identification part of the artificial intelligence based on these rules, based on the findings from comparing fluorescent and light microscopic images acquired with different objectives.

TABLE 3
AI training rules extracted from analyzing microscopic images
Annotation Rules for artificial intelligence training

| | |
|---|---|
| Annotation Rules for oPMNs | 1. Neutrophils are circular, so they can be seen like a circle under the |
| | microscope (1). |
| | 2. Neutrophils can be seen brightly under the microscope. |
| | 3. They range in size from 10-15 μm, so they should not be less or |
| | more than this size (29). |
| | 3. Dead neutrophils have the same size as live ones, but they turned |
| | dark, and they are not bright anymore |
| | 4. Their shape starts to change during the time into amoeboid when |
| | they are activated so that they can extend their pseudopodia (7). |
| | 5. Due to the presence of collagenase and lysosomes in the |
| | cytoplasm, a light microscope's reflection causes neutrophils to be |
| | seen lightly (40). |
| | 6. Neutrophils should not be attached to each other. We consider |
| | them debris because they cannot be counted. |
| | 7. They are multi-lobed nuclei, but their lobes are not seen clearly |
| | under objective 10X. |
| | 8. During degeneration, neutrophil pushes all its granules to the |
| | extracellular matrix (26). |
| Annotation Rules for oral epithelial cells | 1. Epithelial cells are flat with a thin membrane(24) |
| | 2. They are wider than their height(37) |
| | 3. Epithelial cells are round-oval-like in appearance, meaning they |
| | are taller than other cells(24) |
| | 4. Their cytoplasm is dense and homogenous; they are not bright |
| | under the microscope (37) |
| | 5. These cells are typically between 30 to 60 μm in size (39) |
| | 6. They include a large dark spot located in their centre, which is the |
| | nucleus |
| | 7. They may see as fragmented under the microscope because they |
| | have no cell wall; they are very fragile |
| Annotation Rules for debris | 1. Debris will almost be irregular in shape and size |
| | 2. They are mass-like that floating through the petri dish |
| | 3. Accumulation of epithelial or neutrophil cells is considered a |
| | debris |
| | 4. They are usually seen blurry under the microscope |

Implementations of the subject matter and the functional operations described in this specification can be implemented in digital electronic circuitry, in tangibly embodied computer software or firmware, in computer hardware, including the structures disclosed in this specification and their structural equivalents, or in combinations of one or more of them. Implementations of the subject matter described in this specification can be implemented as one or more computer programs, i.e., one or more modules of computer program instructions encoded on a tangible, non-transitory computer-storage medium for execution by, or to control the operation of data processing apparatus. Alternatively or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. The computer-storage medium can be a machine-readable storage device, a machine-readable storage substrate, a random or serial access memory device, or a combination of one or more of them.

A computer program, which may also be referred to or described as a program, software, a software application, a module, a software module, a script, or code can be written in any form of programming language, including compiled or interpreted languages, or declarative or procedural languages, and it can be deployed in any form, including as a stand-alone program or as a module, component, subroutine, or other unit suitable for use in a computing environment. A computer program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a communication network. While portions of the programs illustrated in the various figures are shown as individual modules that implement the various features and functionality through various objects, methods, or other processes, the programs may instead include a number of sub-modules, third-party services, components, libraries, and such, as appropriate. Conversely, the features and functionality of various components can be combined into single components, as appropriate.

The processes and logic flows described in this specification can be performed by one or more programmable computers executing one or more computer programs to perform functions by operating on input data and generating output. The processes and logic flows can also be performed by, and apparatus can also be implemented as, special purpose logic circuitry, e.g., a CPU, a GPU, an FPGA, or an ASIC.

To provide for interaction with a user, implementations of the subject matter described in this specification can be implemented on a computer having a display device, e.g., a CRT (cathode ray tube), LCD (liquid crystal display), LED (Light Emitting Diode), or plasma monitor, for displaying information to the user and a keyboard and a pointing device, e.g., a mouse, trackball, or trackpad by which the user can provide input to the computer. Input may also be provided to the computer using a touchscreen, such as a tablet computer surface with pressure sensitivity, a multi-touch screen using capacitive or electric sensing, or other type of touchscreen. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

The term “graphical user interface,” or “GUI,” may be used in the singular or the plural to describe one or more graphical user interfaces and each of the displays of a particular graphical user interface. Therefore, a GUI may represent any graphical user interface, including but not limited to, a web browser, a touch screen, or a command line interface (CLI) that processes information and efficiently presents the information results to the user. In general, a GUI may include a plurality of user interface (UI) elements, some or all associated with a web browser, such as interactive fields, pull-down lists, and buttons operable by the user. These and other UI elements may be related to or represent the functions of the web browser.

As shown in FIG. 4b, implementations of the subject matter described in this specification can be implemented in a computing system 300 that includes a back-end component, e.g., as a data server 400, or that includes a middleware component, e.g., an application server, or that includes a front-end component, e.g., a client computer 312 having a graphical user interface or a Web browser through which a user can interact with an implementation of the subject matter described in this specification, or any combination of one or more such back-end, middleware, or front-end components 400. The computing system 300 can be interconnected by any form or medium of wireline and/or wireless digital data communication, e.g., a communications network 402. Examples of communication networks include a local area network (LAN), a radio access network (RAN), a metropolitan area network (MAN), a wide area network (WAN), Worldwide Interoperability for Microwave Access (WIMAX), a wireless local area network (WLAN) using, for example, 802.11 a/b/g/n and/or 802.20, all or a portion of the Internet, and/or any other communication system or systems at one or more locations, and free-space optical networks. The network may communicate with, for example, Internet Protocol (IP) packets, Frame Relay frames, Asynchronous Transfer Mode (ATM) cells, voice, video, data, and/or other suitable information between network addresses.

In another implementation, system 200 follows a cloud computing model, by providing an on-demand network access to a shared pool of configurable computing resources (e.g., servers 400, storage, applications, and/or services) that can be rapidly provisioned and released with mini-mal or nor resource management effort, including interaction with a service provider, by a user (operator of a thin client).

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hard-ware and computer instructions.

Benefits, other advantages, and solutions to problems have been described above with regard to specific embodiments. However, the benefits, advantages, solutions to problems, and any element(s) that may cause any benefit, advantage, or solution to occur or become more pronounced are not to be construed as critical, required, or essential features or elements of any or all the claims. As used herein, the terms “comprises,” “comprising,” or any other variations thereof, are intended to cover a non-exclusive inclusion, such that a process, method, article, or apparatus that comprises a list of elements does not include only those elements but may include other elements not expressly listed or inherent to such process, method, article, or apparatus. Further, no element described herein is required for the practice of the invention unless expressly described as “essential” or “critical.”

The preceding detailed description of exemplary embodiments of the invention makes reference to the accompanying drawings, which show the exemplary embodiment by way of illustration. While these exemplary embodiments are described in sufficient detail to enable those skilled in the art to practice the invention, it should be understood that other embodiments may be realized and that logical and mechanical changes may be made without departing from the spirit and scope of the invention. For example, the steps recited in any of the method or process claims may be executed in any order and are not limited to the order presented. Thus, the preceding detailed description is presented for purposes of illustration only and not of limitation, and the scope of the invention is defined by the preceding description, and with respect to the attached claims.

### REFERENCES

1. Kinane, D. F., Stathopoulou, P. G. & Papapanou, P. N. Periodontal diseases. Nature Reviews Disease Primers 3, 1-14 (2017).
2. Periodontal Disease,<https://www.cdc.gov/oralhealth/conditions/periodontal-discase.html//periodontal>(2013).
3. Nazir, M. A. Prevalence of periodontal disease, its association with systemic diseases and prevention. International journal of health sciences 11, 72 (2017).
4. Taylor, J. J., Preshaw, P. M. & Lalla, E. A review of the evidence for pathogenic mechanisms that may link periodontitis and diabetes. Journal of clinical periodontology 40, S113-S134 (2013).
5. Stöhr, J., Barbaresko, J., Neuenschwander, M. & Schlesinger, S. Bidirectional association between periodontal disease and diabetes mellitus: a systematic review and meta-analysis of cohort studies. Scientific Reports 11, 1-9 (2021).
6. Hajishengallis, G. & Chavakis, T. Local and systemic mechanisms linking periodontal disease and inflammatory comorbidities. Nature Reviews Immunology, 1-15 (2021).
7. Leech, M. T. & Bartokl, P. M. The association between rheumatoid arthritis and periodontitis. Best practice d research Clinical rheumatology 29, 189-201 (2015).
8. Xu, S., Zhang, G., Guo, J. f. & Tan, Y. h. Associations between osteoporosis and risk of periodontitis: A pooled analysis of observational studies. Oral diseases 27, 357-369 (2021).
9. Zardawi, F., Gul, S., Abdulkareem, A., Sha, A. & Yates, J. Association between periodontal disease and atherosclerotic cardiovascular diseases: revisited. Frontiers in Cardiovascular Medicine 7(2020).
10. Gibbons, G. W. & Shaw, P. M. in Seminars in vascular surgery. 89-92 (Elsevier).
11. Yoo, J. J., Kim, D. W., Kim, M. Y., Kim, Y. T. & Yoon, J. H. The effect of diabetes on tooth loss caused by periodontal disease: A nationwide population-based cohort study in South Korea. Journal of periodontology 90, 576-583 (2019).
12. Wilcox, M. E. et al. Oral neutrophils are an independent marker of the systemic inflammatory response after cardiac bypass. Journal of Inflammation 11, 1-11 (2014).
13. Forster, C., Aboodi, G., Lipton, J. & Glogauer, M. A non-invasive oral rinse assay predicts bone marrow engraftment and 6 months prognosis following allogeneic hematopoietic stem cell transplantation. Journal of oral pathology & medicine 41, 165-170 (2012).
14. Lieschke, G. J. et al. Studies of oral neutrophil levels in patients receiving G-CSF after autologous marrow transplantation. British journal of haematology 82, 589-595 (1992).
15. Cheretakis, C., Leung, R., Sun, C. X., Dror, Y. & Glogauer, M. Timing of neutrophil tissue repopulation predicts restoration of innate immune protection in a murine bone marrow transplantation model. Blood 108, 2821-2826 (2006).
16. Wright, D., Meierovics, A. & Foxley, J. Assessing the delivery of neutrophils to tissues in neutropenia. (1986).
17. Akpek, G., Knight, R. D. & Wright, D. G. Use of oral mucosal neutrophil counts to detect the onset and resolution of profound neutropenia following high-dose myelosuppressive chemotherapy. American journal of hematology 72, 13-19 (2003).
18. Domnich, M. et al. Oral Neutrophils: Underestimated Players in Oral Cancer. Frontiers in Immunology 11, doi:10.3389/fimmu.2020.565683 (2020).
19. Huda, S. et al. Oral neutrophil levels: a screening test for oral inflammatory load in pregnancy in a medical setting. Journal of periodontology 86, 72-81 (2015).
20. Davison, G. & Jones, A. W. Oral neutrophil responses to acute prolonged exercise may not be representative of blood neutrophil responses. Applied Physiology, Nutrition, and Metabolism 40, 298-301 (2015).
21. Cheretakis, C., Dror, Y. & Glogauer, M. A noninvasive oral rinse assay to monitor engraftment, neutrophil tissue delivery and susceptibility to infection following HSCT in pediatric patients. Bone marrow transplantation 36, 227-232 (2005).
22. Hirschfeld, J. Neutrophil subsets in periodontal health and disease: a mini review. Frontiers in Immunology 19 (2019).
23. Barreiro, O. & Sánchez-Madrid, F. Molecular basis of leukocyte-endothelium interactions during the inflammatory response. Revista Española de Cardiologia (English Edition) 62, 552-562 (2009).
24. Varki, A. Sialic acids in human health and disease. Trends in molecular medicine 14, 351-360 (2008).
25. Wigcek, A. E., Gozdecka, A., Jurak, M., Przykaza, K. & Terpilowski, K. Wettability of plasma modified glass surface with bioglass layer in polysaccharide solution. Colloids and Surfaces A: Physicochemical and Engineering Aspect 551, 185-194 (2018).
26. Marques, A., Reis, R. & Hunt, J. The effect of starch-based biomaterials on leukocyte adhesion and activation in vitro. Journal of Materals Science: Materials in Medicine 16, 1029-1043 (2005).
27. Borges, L. et al. Neutrophil migration and adhesion molecule expression after acute high-intensity street dance exercise. Journal of immunology research 2918 (2018).
28. Rijkschroeff, P. et al. Dentistry and OMICS: Transcriptome Dynamics of an Oral Ecosystem as Measured by Changes in Oral Polymorphonuclear Neutrophils in Experimental Gingivitis. OMICS: A Journal of Integrative Biology 24, 531-540 (2020).
29. Landzberg, M., Doering, H., Aboodi, G., Tenenbaum, H. & Glogauer, M. Quantifying oral inflammatory load: oral neutrophil counts in periodontal health and disease. Journal of periodontal research 50, 330-336 (2015).
30. Alam, M. M. & Islam, M. T. Machine learning approach of automatic identification and counting of blood cells. Healthcare technology letters 6, 103-108 (2019).
31. Kutlu, H., Avci, E. & Özyurt, F. White blood cells detection and classification based on regional convolutional neural networks. Medical hypotheses 135, 109472 (2020).
32. Patil, A., Patil, M. & Birajdar, G. White blood cells image classification using deep learning with canonical correlation analysis. IRBM(2020).
33. Hegde, R. B., Prasad, K., Hebbar, H. & Singh, B. M. K. Comparison of traditional image processing and deep learning approaches for classification of white blood cells in peripheral blood smear images. Biocybernetics and Biomedical Engineering 39, 382-392 (2019).
34. Sahlol, A. T., Kollmannsberger, P. & Ewees, A. A. Efficient classification of white blood cell leukemia with improved swarm optimization of deep features. Scientific reports 10, 1-11 (2020).
35. Lin, T.-Y. et al. in European conference on computer vision. 740-755 (Springer).
36. Teuschl, A. H. et al. Enhanced cell adhesion on silk fibroin via lectin surface modification. Acta biomaterialia 10, 2506-2517 (2014).
37. Howlett, C. R., Evans, M. D., Walsh, W. R., Johnson, G. & Steele, J. G. Mechanism of initial attachment of cells derived from human bone to commonly used prosthetic materials during cell culture. Biomaterials 15, 213-222 (1994).
38. Cunningham, R., Carr, M., Bagley, E., Gordon, S.& Greene, R. Hydrophilic foams containing corn products for horticultural use. Journal of applied polymer science 51, 1311-1317 (1994).
39. Kaplan, S., Basford, R., Jeong, M. & Simmons, R. Biomaterial—neutrophil interactions: Dysregulation of oxidative functions of fresh neutrophils induced by prior neutrophil-biomaterial interaction. Journal of Biomedical Materials Research: An Official Journal of The Society for Biomaterials and The Japanese Society for Biomaterials 30, 67-75 (1996).
40. Hampton, M. B., Kettle, A. J. & Winterbourn, C. C. Involvement of superoxide and myeloperoxidase in oxygen-dependent killing of Staphylococcus aureus by neutrophils. Infection and immunity 64, 3512-3517 (1996).
41. Diamond, M. S., Staunton, D. E., Marlin, S. D. & Springer, T. A. Binding of the integrin Mac-1 (CD11b/CD18) to the third immunoglobulin-like domain of ICAM-1 (CD54) and its regulation by glycosylation. Cell 65, 961-971 (1991).
42. Kelm, M. et al. Regulation of neutrophil function by selective targeting of glycan epitopes expressed on the integrin CD11b/CD18. The FASEB Journal 34, 2326-2343 (2020).
43. Nadzam, G. S., De La Cruz, C., Greco, R. S. & Haimovich, B. Neutrophil adhesion to vascular prosthetic surfaces triggers nonapoptotic cell death. Annals of surgery 231, 587 (2000).
44. Yakubenko, V. P., Lishko, V. K., Lam, S. C.-T. & Ugarova, T. P. A molecular basis for integrin αMβ2 ligand binding promiscuity. Journal of Biological Chemistry 277, 48635-48642(2002).
45. Zimmerman, G. & McIntyre, T. Neutrophil adherence to human endothelium in vitro occurs by CDw18 (Mol, MAC-1/LFA-1/GP 150, 95) glycoprotein—dependent and—independent mechanisms. The Journal of clinical investigation 81, 531-537 (1988).
46. Liu, L., Elwing, H., Karlsson, A., Nimeri, G. & Dahlgren, C. Surface—related triggering of the neutrophil respiratory burst. Characterization of the response induced by IgG adsorbed to hydrophilic and hydrophobic glass surfaces. Clinical & Experimental Immunology 109, 204-210 (1997).
47. Wardle, E. N. Assessment of neutrophil function—I. Postgraduate medical journal 62, 997 (1986).
48. Labeling. Git code, <https://github.com/tzutalin/labelling>(2015).
49. Redmon, J., Divvala, S., Girshick, R. & Farhadi, A. in Proceedings of the IEEE conference on computer vision and pattern recognition. 779-788.
50. Redmon, J. & Farhadi, A. in Proceedings of the IEEE conference on computer vision and pattern recognition. 7263-7271.
51. Redmon, J. & Farhadi, A. Yolov3: An incremental improvement. arXiv preprint arXiv:1804.02767 (2018).
52. Bochkovskiy, A., Wang, C.-Y. & Liao, H.-Y. M. Yolov4: Optimal speed and accuracy of object detection. arXiv preprint arXiv:2004.10934 (2020).
53. Lin, T.-Y. et al. in Proceedings of the IEEE conference on computer vision and pattern recognition. 2117-2125.
54. Liu, S., Qi, L., Qin, H., Shi, J. & Jia, J. in Proceedings of the IEEE conference on computer vision and pattern recognition. 8759-8768.
55. Wang, C.-Y. et al. in Proceedings of the IEEE/CVF conference on computer vision and pattern recognition workshops. 390-391.
56. Takeichi, M. & Okada, T. Roles of magnesium and calcium ions in cell-to-substrate adhesion. Experimental cell research 74, 51-60 (1972).
57. Ribeim, D. et al. Calcium pathways in human neutrophils—the extended effects of thapsigargin and ML-9i. Cells 7, 204 (2018).
58. Schaff, U. Y. et al. Calcium flux in neutrophils synchronizes β 2 integrin adhesive and signaling events that guide inflammatory recruitment. Annals of biomedical engineering 36, 632-646 (2008).
59. Joseph, N., Reicher, B. & Barda-Saad, M. The calcium feedback loop and T cell activation: how cytoskeleton networks control intracellular calcium flux. Biochimica et Biophysica Acta (BBA)—Biomembranes 1838, 557-568 (2014).
60. Carlson, J. et al. Stability of African swine fever virus in soil and options to mitigate the potential transmission risk. (2020).
61. Weldon, B. A. et al. Using primary organotypic mouse midbrain cultures to examine developmental neurotoxicity of silver nanoparticles across two genetic strains. Toxicology and applied pharmacology 354, 215-224 (2018).
62. Dempsey, S. G. et al. A novel chemotactic factor derived from the extracellular matrix protein decorin recruits mesenchymal stromal cells in vitro and in vivo. PloS one 15, e0235784 (2020).

The invention claimed is:

1. A method of separating neutrophils from a heterogeneous cell suspension comprising the steps of:
receiving salivary samples;
treating a surface of a receptacle treated with oxygen plasma to increase the hydrophilic properties of the surface that subsequently increase cell-surface adhesion;
transferring the salivary samples to the receptacle;
adding phosphate-buffered saline (PBS) to levitate epithelial cells and separate them from oral polymorphonuclear neutrophils (oPMNs) in saliva;
removing the levitated epithelial cells after a delay time (at), whereby the oPMNs are attached to the surface of the receptacle.

2. The method of claim 1, wherein the surface of the receptacle is treated with com-starch to increase the hydrophilic properties of the surface that subsequently increase cell-surface adhesion.

3. The method of claim 1, wherein the oxygen plasma comprises a 70% power intensity.

4. The method of claim 2, wherein the oxygen plasma increases the adhesion of oPMNs surrounded by hydrophilic CD markers.

5. The method of claim 3, wherein the PBS washes and weakens the bonding between the epithelial cells or debris and a surface of the petri-dishes, and remove dead, non-adhesive, and lose cells, including cellular particles.

6. The method of claim 1, wherein adhesion properties of the neutrophils are dependent on a plurality of parameters comprising at least one of delay time, oxygen plasma, corn-starch, CaCl2), PBS, and surface materials.

7. The method of claim 1, comprising a further steps of:
capturing microscopic images of the surface with the isolated oPMNs; and
counting the oPMNs level in each of the salivary samples.

8. The method of claim 8, wherein the number of isolated oPMNs

9. The method of claim 1, wherein oPMNs level is dependent on a plurality of parameters comprising at least one of delay time, oxygen plasma, corn-starch, CaCl2, PBS, and surface materials.

10. An automated method of separating neutrophils from a heterogeneous cell suspension comprising the steps of:
receiving a salivary sample in a test tube;
adding phosphate-buffered saline (PBS) to the test tube to create a mixture of the salivary sample and the PBS;
with a programmed microcontroller system, causing a directional pump to turn on and deliver the mixture to a Petri dish, and causing the directional pump to turn off; and after a predefined delay time (at) causing the directional pump to turn on and remove the solution from the Petri dish and return the solution into the test tube, whereby the neutrophils are attached to the surface of the Petri dish; and wherein the Petri dish is placed on a rotatable platform;
transferring the salivary samples to the test tube;
with the programmed microcontroller system, causing a servomotor to rotate the platform with the Petri dish;
with a microscope and an image capture device, capturing multiple microscopic images as the Petri dish rotates.

11. The automated method of claim 10, wherein the programmed microcontroller system comprises a graphical interface system to control the directional pump, the servomotor and the image capture device.

12. The automated method of claim 11, wherein the programmed microcontroller system comprises a memory with first instructions executable by a processor to at least cause the directional pump to turn on in first direction for a first predefined time (Tₒₙ₁), then cause the directional pump to turn off for a second predefined time (T_{off1}), cause the directional pump to turn on in a second direction for a second predefined time (Tₒₙ₂), then cause the directional pump to turn pump turn off.

13. The automated method of claim 12, wherein the programmed microcontroller system comprises second instructions executable by the processor to at least cause the servomotor to rotate a predefined angle (α), and cause the image capture device to capture one microscopic image, then cause the servomotor to rotate for about a and cause the image capture device to capture another microscopic image, and repeating this process to capture a predetermined number (k) of microscopic images.

14. The automated method of claim 13, wherein each microscope image comprises Nᵢ(i=1 to k) oral neutrophils.

15. The automated method of claim 14, comprising a step of counting the number of oral neutrophils.

16. The automated method of claim 14, wherein the the number of cells in saliva is equal to the average of Nᵢ multiplied by a surface area of the petri-dish.

17. A system for separating neutrophils from a heterogeneous cell suspension comprising:
a mixture of a salivary sample and phosphate-buffered saline (PBS) in a test tube;
a programmed microcontroller system;
a directional pump controllable by the programmed microcontroller system to turn on and off, and remove the solution from the Petri dish after a predefined delay time (σt), and return the solution into the test tube; and wherein the Petri dish is placed on a rotatable platform;
a servomotor controllable by the programmed microcontroller system to rotate the platform with the Petri dish;
a microscope and an image capture device controllable by the programmed microcontroller system to capturing multiple microscopic images as the Petri dish rotates.

18. The system of claim 17, wherein the programmed microcontroller system comprises a memory with first instructions executable by a processor to at least cause the directional pump to turn on in first direction for a first predefined time (Tₒₙ₁), then cause the directional pump to turn off for a second predefined time (T_{off1}), cause the directional pump to turn on in a second direction for a second predefined time (Tₒₙ₂), then cause the directional pump to turn pump turn off.

19. The system of claim 17, wherein the programmed microcontroller system comprises second instructions executable by the processor to at least cause the servomotor to rotate a predefined angle (α), and cause the image capture device to capture one microscopic image, then cause the servomotor to rotate for about a and cause the image capture device to capture another microscopic image, and repeating this process to capture a predetermined number (k) of microscopic images.

20. The system of claim 18, wherein each microscope image comprises Nᵢ (i=1 to k) oral neutrophils.

21. The system of claim 18, comprising a step of counting the number of oral neutrophils.

22. The system of claim 18, wherein the the number of cells in saliva is equal to the average of Nᵢ multiplied by a surface area of the petri-dish.

23. A system comprising:
an imaging system for capturing at least one microscopic image of a surface comprising a plurality of isolated neutrophils;
a computer system comprising a hardware processor and a memory device on which instructions are encoded to cause the hardware processor to perform the operations of:
receiving the at least one microscopic image;
extracting at least one feature vector set from the image datasets for input into a machine learning architecture;
generating a machine learning model iteratively trained to detect each of the plurality of isolated neutrophils within the at least one microscopic image;
annotating the at least one microscopic image by placing a bounding box around and generating image datasets;
and applying the trained machine learning model to classify each of the plurality of isolated neutrophils appearing within the at least one microscopic image;
based on the classification, predicting the number of the plurality of isolated neutrophils within the at least one microscopic image.

24. The system of claim 23, wherein the machine learning architecture comprises a convolutional neural network (CNN) architecture.

25. The system of claim 23, wherein each bounding box comprises a class probability and regression problem information comprising a location and a topography of an object of interest (x, y, w, h) within the microscopic image.

26. The system of claim 25, wherein the object of interest within the microscopic image is an oral neutrophil.
